(19) United States
(12) Patent Application Publication
(10) Pub. No.: US 2018/0071068 A1
(43) Pub. Date: Mar. 15, 2018

(54) STABILIZATION DEVICE FOR ANIMALS

(71) Applicants: Andreas THIEME, Pulsnitz (DE); Torsten URSINUS, Dresden (DE)

(72) Inventors: Andreas THIEME, Pulsnitz (DE); Torsten URSINUS, Dresden (DE)

(21) Appl. No.: 15/602,073

(22) Filed: May 22, 2017

(30) Foreign Application Priority Data
Jun. 30, 2016 (DE) 20 2016 004 112.7

(51) Int. Cl. A61D 3/00; A61B 5/11; A61F 7/00

(52) U.S. Cl. CPC A61D 3/00; A61B 5/1112; A61F 7/0097; A61F 7/007; A61F 2007/0075; A61F 2007/0086; A61B 2503/40

(57) ABSTRACT

The invention relates to a stabilising device for animals for medical treatment, for relieving strain, stabilising, securing, and to enable them to be lifted horizontally and carried, comprising various components. These include a flat belly element, which is designed to be attached in the belly region and has a flexible, anatomically adaptable supporting area and at least four connector elements with first closure means arranged thereon. The components of the stabilising device further include a flat back element which is designed to be attachable in the back region, and has at least one means for stabilising at least partially to the longitudinal axis, and at least four corresponding second closure means. The at least four connector elements are arranged on the belly element at a distance from each other mirror symmetrically to the longitudinal axis of the belly element. The belly element is further designed to be detachably connectable to the back element via the connector elements with the first closure means arranged thereon and the corresponding second closure means, wherein the number of first closure means is equal to the number of corresponding second closure means.

The invention relates to a stabilising device for animals for medical treatment, for relieving strain, stabilising, securing, and to enable them to be lifted horizontally and carried.

The stabilising device for animals comprises various components such as a flat belly element with a flexible, anatomically adaptable supporting area and a flat back element with at least one stabilising means. The components are designed to be detachably connected to each other via connecting elements and connecting means.

Harnesses, such as dog harnesses, for example, are used by the owner or user to guide, train and support the dog. As a rule, they consist of a strap over the chest and back region, or over the chest or back region only.

Document U.S. Pat. No. 4,559,906 A discloses a harness for dogs and cats. Such a harness includes straps which are wrapped around the belly and back respectively, and a front strap that passes across the animal's lower neck or chest region. The animal can be lifted and carried with a rigid handle which is attached to the straps in the back region. The design of the handle distributes the lifting forces evenly over the body, subjecting the animal to less stress.

Document U.S. Pat. No. 7,325,516 B2 describes a carrying system for supporting weak or sick animals, particularly when climbing stairs. The carrying system comprises a vest which is fastened detachably around the animal's body with hook and loop fastenings. The vest covers the animal's chest, belly, shoulder and back region. Variously configured handles and straps are affixed to the vest. In this case, particular emphasis is placed on lifting the hips, particularly for sick or injured animals.

Document U.S. Pat. No. 8,336,506 B2 discloses a hip lift harness for dogs. This comprises a chest member and a hip member, which extend separately around the rump and are connected to each other via straps. Both members are also furnished with handles, by which the dog can be lifted into a car, for example.

In U.S. Pat. No. 9,320,260 B2 also, the hip lift harness disclosed in U.S. Pat. No. 8,336,506 B2 is expanded with embodiments in which a line is attached between a D-ring provided on the chest element and the handle on the hip element, and is designed as a carrying strap specifically for larger animals.

The straps and harnesses disclosed in the prior art are particularly suitable for smaller mammals such as dogs and cats.

Moreover, the straps and harnesses disclosed have significant shortcomings in terms of an anatomically protective carrying function, since they either constrict the belly, muscles and blood vessels, place undue stress on the spine, or are not adaptable appropriately to the animal's body.

It would therefore be extremely desirable to provide a harness that is designed to enable the animal to be held in place and supported in a way that is comfortable for the animal both in a resting position and when it is being carried and lifted. The harness should also be suitable for use with larger mammals.

The object is to provide a harness for animals that does not share the drawbacks of the prior art.

At the same time, the musculoskeletal system of an animal which has been injured, undergone surgery or is advanced in age should be treated as gently as possible. Additionally, the harness should function to subject the animal to as little physical stress as possible when it is lifted and carried while ensuring stable support and secure fastening in the harness without straining, constricting or restricting it.

The handle should also enable animal's owner to use the harness comfortably and conveniently for lifting and carrying the animal or when taking it for a walk.

The object is solved according to the invention with a stabilising device for animals as defined in the independent claims. Advantageous embodiments of the invention are described in the dependent claims.

A first aspect of the invention relates to a stabilising device for animals comprising various components for medical treatment, for relieving strain, stabilising, securing, and to enable them to be lifted horizontally and carried. The components include a flat belly element which is designed to be attached in the belly region and a flexible, anatomically adaptable supporting area having at least four connector elements with first closure means arranged thereon. The stabilising device further comprises a flat back element for the components of the stabilising device, which back element is designed to be applied in the back region, and includes at least one stabilising means aligned at least in sections to the longitudinal axis, and at least four second closure means. In this context, the at least four connector elements are arranged at a distance from each other on the belly element mirror symmetrically to the longitudinal axis of the belly element. The belly element is further designed to be detachably connectable to the back element via the connector elements with the first closure means arranged thereon and the corresponding second closure means, wherein the number of first closure means is equal to the number of corresponding second closure means.

One embodiment functions as a stabilising device for animals. In one embodiment, the term animal is understood to mean any mammal. In one embodiment, the term animal is understood to mean any living creature (with the exception of Man). In a further embodiment, the term animal is understood to mean any animal with four legs. An animal with four legs is also referred to as a quadruped. These also include working and farm animals. The term quadruped further embraces dogs, cats, horses, particularly racehorses and sport horses, and camels.

In the following text, the description stabilising device for animals will be shortened to stabilising device.

In one embodiment, the stabilising device comprises different components.

In one embodiment, the stabilising device includes at least partly reflective elements. The reflective elements are advantageous affixed to the surface of the stabilising device facing outwards so that the carrier of the stabilising device is easily visible in traffic even in dark or poor light conditions due to the reflection.

In one embodiment, the components of the stabilising device comprise a belly element and a back element. In a preferred embodiment, the components of the stabilising device comprise a belly element, a back element and a front part. In this way, the entire rump of the animal is advantageous surrounded by the stabilising device.

In one embodiment, the front part is in the form of a standard commercial harness.

In a preferred embodiment, the front part includes a chest element and a shoulder element. This allows the stabilising device according to the invention to be used in conjunction with a standard commercial strap or harness. In a further embodiment, the components of the stabilising device comprise a belly element, a back element, a chest element and a shoulder element.

In one embodiment, the stabilising device is designed such that it may be completely disassembled into its individual components. In a further embodiment, the straps are completely detachable from the individual components.

In one embodiment, the stabilising device further comprises connector elements. In one embodiment, the connector elements are in the form of belts or straps or as belt straps.

In one embodiment, the components and/or the connector elements of the stabilising device may be designed individually with variable and various dimensions. In one embodiment, the components and/or the connector elements of the stabilising device may be combined with each other. In one embodiment, the components and/or the connector elements of the stabilising device may be designed individually with variable and various dimensions and may be combined with each other.

The stabilising device is thus advantageously adaptable individually to the respective animal and its necessities and needs. Accordingly, due account is taken of the quadruped's weight, size and anatomy as well as its capacity to withstand stress.

In a preferred embodiment, the stabilising device has a modular construction. A modular construction is understood to mean that variously designed components and connector elements of the stabilising device are capable of being combined.

The stabilising device according to the invention is designed so that it may be fitted on the animal and removed again easily and quickly.

According to the invention, the stabilising device comprises a belly element. The belly element is preferably designed to be fitted in the animal's belly region. For the purposes of the invention, the animal's belly region is understood to be its abdomen.

According to the invention, the belly element is of substantially flat design. Within the meaning of the invention, a substantially flat design of the belly element is understood to mean a belly element that extends over extensive areas of the belly. In one embodiment, the belly element extends over at least 60%, preferably at least 70%, most particularly preferably at least 80% of the animal's belly region.

In a preferred embodiment, the belly element of the stabilising device has a cutout for the sex organ of male animals such as male dogs. In one embodiment, the belly element has a cutout for the sex organ of male animals such as male dogs. This advantageously also enables the animal to urinate easily and increases the carrying comfort of the stabilising device.

According to the invention, the belly element has a flexible, anatomically adaptable supporting area. For purposes of the invention, the phrase anatomically adaptable means that the belly element is flexible enough to fit snugly against the animal's belly region. This increases carrying comfort. Also advantageously, such a design of the supporting area serves to ensure that the animal's musculature, joints and vascular system for example are not subjected to undue strain. In this way, the animal's musculoskeletal system is treated gently.

In one embodiment, the supporting area of the belly element is adapted individually to the animal's belly region. In one embodiment, the supporting area of the belly element is pear-shaped.

According to the invention, at least four connector elements are arranged on the belly element. In an alternative embodiment, at least two connector elements are arranged on the belly element.

According to the invention, at least four connector elements with first closure means attached thereto are arranged on the belly element. In a further embodiment, four connector elements are arranged on the belly element. In one embodiment, the connector elements are arranged punctually, i.e. at different points on the belly element. In one embodiment, the connector elements are arranged on the belly element at a distance from each other. In one embodiment, the connector elements arranged on the belly element do not touch each other. This advantageously ensures a high degree of stability for the stabilising device.

In one embodiment, the connector elements are arranged on the belly element so as to be attached non-detachably, by sewing for example.

In one embodiment, the connector elements are arranged on the belly element mirror symmetrically about the longitudinal axis of the belly element. In a preferred embodiment, the at least four connector elements are arranged on the belly element at a distance from each other mirror symmetrically to the longitudinal axis of the belly element. In this context, the longitudinal axis of the belly element is the notional centrally arranged line in the running direction of the animal. In a preferred embodiment, two connector elements are arranged on the left side of the belly element and two connector elements are arranged on the right side of the belly element relative to the longitudinal axis. The symmetrical arrangement of the connector elements when the belly and back elements are connected advantageously ensures a stable, firm and slip-proof grip on the stabilising device.

In one embodiment, the stabilising device comprises at least four connector elements. In an alternative embodiment, the stabilising device comprises at least two connector elements. In a preferred embodiment, the stabilising device comprises four connector elements.

In a preferred embodiment, first closure means are arranged on the at least four connector elements of the belly element. In one embodiment, at least four first closure means are arranged on the at least four connector elements of the belly element.

In one embodiment, the number of connector elements on the belly element is equal to the number of the first closure means, so that each connector element on the belly element advantageously has a first closure means. In one embodiment, a first closure means is arranged on each connector element of the belly element. In one embodiment four first closure means are arranged on the four connector elements of the belly element. In an alternative embodiment, two first closure means are arranged on the two connector elements of the belly element.

In one embodiment, the first closure means of the belly element comprise mechanical closure means such as buckles, particularly roller buckles, double pin buckles, triple pin buckles, ladder-lock buckles, sliding buckles, clamping buckles, click buckles (quick fit or click lock, also called click clasps), quick release buckles or interlocking buckles. In a further embodiment, the first closure means of the belly element comprise mechanical closure means such as toggle closures as toggle closures, hook and loop closures, press studs or zip fasteners. In a further embodiment, the first closure means of the belly element comprise mechanical closure means such as plug locks. In a further embodiment, the first closure means of the belly element include sliding bars, with which the length of the connector elements with the back element may advantageously be adjusted.

In one embodiment, the connector elements are at least partly arranged on the edges of the belly element. In a further embodiment, the connector elements are arranged on the edges of the belly element, mirror symmetrically to the longitudinal axis of the belly element. In a further embodiment, all connector elements are arranged on the edges of the belly element.

In one embodiment, the stabilising device comprises a back element. According to the invention, the back element is designed so as to be placeable on the animal's back.

The term back element is also understood to mean a back section, for example.

According to the invention, the back element is of flat design. Within the meaning of the invention, a flat design of the belly element is understood to mean a back element that extends over extensive areas of the back.

In one embodiment, the back element is designed for stabilising effect. In one embodiment, the back element has a stabilising effect and is rigid in the region of the spine. In this way, the spine is advantageously supported and prevented from sagging. In one embodiment, the stabilising structure of the back element is assured with a stabilising insert.

In one embodiment, the supporting area of the back element is adapted to the individual animal's back region.

For the purposes of the invention, the back element includes at least one stabilising means, at least a portion of which extends to the longitudinal axis. For the purposes of the invention, the term stabilising means is also understood to mean a back panel for example.

In one embodiment, at least a portion of the back element to the longitudinal axis has a stabilising means. In an alternative embodiment, at least a portion of the back element to the longitudinal axis has a plurality of stabilising means. In this case, the stabilising means are advantageously arranged parallel and at a distance from each other to lend good stability of the back element.

In one embodiment, the stabilising means extends continuously to the longitudinal axis of the back element. In an alternative embodiment, multiple stabilising means extend continuously to the longitudinal axis of the back element. In this case, the stabilising means are advantageously arranged parallel to the longitudinal axis and at a distance from each other to assure good stability of the back element.

The longitudinal axis of the back element represents the notional centrally arranged line on the back element which extends in the running direction of the animal. The longitudinal axis of the back element extends along the animal's spine.

In a further embodiment, the stabilising means in the back element is a panel or a brace. In one embodiment, the stabilising means in the back element is introduced into a cutout provided for this purpose on the upper side of the back element. In a further embodiment, the stabilising means in the back element is secured inside the back element by sewing for example. In a further embodiment, the stabilising means of the back element is sewn underneath a connector element of the back element.

In one embodiment, the stabilising means in the back element is a metal brace or a plastic brace, wherein the plastic brace presents the further advantage of reducing the weight of the back element.

Inserts and seams serve to stabilise the back element further.

In one embodiment, the back element is equipped with second closure means corresponding to the first closure means on the belly element. In one embodiment, the second closure means represent the corresponding counterpieces to the first closure means, so that the first and second closure means are advantageously designed to be detachably connectable to each other in positive locking manner.

In one embodiment, the first and second closure means are made from the same material. In an alternative embodiment, the first and second closure means are made from different materials. Examples of materials that are used for the first and second closure means are metals and advantageously plastics.

According to the invention, the number of the first closure means on the belly element is equal to the number of corresponding second closure means on the back element. In one embodiment, at least four corresponding second closure means are arranged on the back element for the first closure means on the belly element. In a further embodiment, four corresponding second closure means are arranged on the back element for the first closure means on the belly element.

In this context, the corresponding second closure means are advantageously arranged on the back element in such manner that they are easy for a user to connect them with the first closure means on the belly element. Both the first closure means and the corresponding second closure means are arranged on the belly and back elements so as to be easily accessible for the user.

In one embodiment, the belly element is designed to be detachably connectable to the back element.

According to the invention, the belly element is detachably connectable to the back element by means of the connector elements located on the belly element with the first closure means attached thereto and the corresponding second closure means which are arranged on the back element. The closure means on the belly element is designed to be connectable to the corresponding second closure means on the back element in such manner that it is mechanically detachable.

In a further embodiment, the connector elements of the belly element and the corresponding second closure means on the back element are constructed as hook and loop closures which can be detachably connected to each other.

When the belly element and the back element are connected to each other, in a preferred embodiment the at least four connector elements extend along anatomically suitable positions of the animal's rump. In an alternative embodiment, the at least two connector elements in a preferred embodiment extend along anatomically suitable positions of the animal's rump, when the belly element and the back element are connected to each other.

According to the invention, anatomically suitable positions are understood to be those positions which do not hinder or hurt the animal when it moves. These include for example the course of the connector elements along suitable ribs, particularly in the region of the third, fourth or fifth rib. Advantageously, the route of the connector element in the region of the third, fourth or fifth rib offers the greatest stability when the stabilising device is fitted. When connected, the connector elements also pass between the belly element and the back element without straining the muscles or constricting any blood vessels.

In one embodiment, the stabilising device includes at least one detachable carrying element. This may then be used advantageously to lift the animal up or carry it. In a preferred embodiment, at least one detachable carrying element may be inserted in the first retaining devices.

In one embodiment, the carrying element is detachably connected to the stabilising device. In one embodiment, the carrying element is arranged so as to be detachably connected to the stabilising device via first retaining devices.

In a preferred embodiment, the stabilising device has at least two first retaining devices.

In a preferred embodiment, the at least two first retaining devices are arranged on the back element and/or on the shoulder element of the stabilising device. In one embodiment, the back element and/or the shoulder element has/have more than two first retaining devices arranged at a distance from each other.

In one embodiment, the at least two first retaining devices are arranged at a distance from each other. In one embodiment the at least two first retaining devices are arranged to the longitudinal axis of the back element and/or the longitudinal axis of the shoulder element, and thus along the spine.

In one embodiment, the at least two first retaining devices are connected to the back element and/or the should element non-detachably. In one embodiment, a first retaining device is connected to the back element and/or the should element non-detachably. For this purpose, in one embodiment a part of the back element and/or the shoulder element is in the form of a belt strap, wherein the belt strap is sewn in such manner that is has a loop into which the first retaining device may be inserted. The belt strap itself is connected non-detachably to the back element and/or the shoulder element, by sewing for example, and lies closely against the back element and/or the shoulder element.

In one embodiment, two first retaining devices are each arranged non-detachably on the back element of the stabilising device. In a further embodiment, two first retaining devices are each arranged non-detachably on the shoulder element of the stabilising device. In an alternative embodiment, at least one first retaining device is arranged on the back element and at least one first retaining device is each arranged on the shoulder element, each being non-detachable. In an alternative embodiment, one first retaining device is arranged on the back element and one first retaining device is arranged on the shoulder element of the stabilising device, each being non-detachable.

In one embodiment, the two first retaining devices are arranged on the two ends in the longitudinal axis of the back element and/or of the shoulder element, each being non-detachable. In one embodiment, two first retaining devices are arranged on the two ends to the longitudinal axis of the back element and two first retaining devices are arranged on the two ends to the longitudinal axis of the shoulder element, each being non-detachable.

In a further alternative embodiment, two first retaining devices are arranged on the back element and one first retaining device is arranged on the shoulder element, each being non-detachable. With these arrangements, it is advantageously possible to insert the carrying element in various ways, so that the animal may be lifted up either at the rear part or completely.

In a further alternative embodiment, a first retaining device is arranged on the back element and two first retaining devices are arranged on the shoulder element, each being non-detachable. With these arrangements, it is advantageously possible to insert the carrying element in various ways, so that the animal may be lifted up either at the front part or completely.

In a further alternative embodiment, two first retaining devices are arranged on the back element and two first the carrying element are arranged on the shoulder element, each being non-detachable. With these arrangements, it is advantageously possible to insert the carrying element in various ways, so that the animal may be lifted up either at the rear part or the front part or completely.

In one embodiment, the first retaining devices of the stabilising device are in the form of rings, particularly o-rings, eyelets or D-rings for fastening the carrying handles. In one embodiment, the first retaining device is made from a sturdy material such as metal, a metal alloy or plastic.

In one embodiment, the carrying element is in the form of a handle. In a further embodiment, he carrying element is in the form of a carrying handle or lanyard, wherein the lanyard is advantageously elastic. In one embodiment, the carrying element has two ends. The carrying element is advantageous made from plastic or sturdy textile material.

In one embodiment, the carrying element has provision for at least one detachable connector in the form of a second retaining device, wherein the second retaining device is designed to be connectable to the first retaining device. In one embodiment, the carrying element has provision for two detachable connectors in the form or two second retaining devices to the first retaining devices.

In a preferred embodiment, the detachable carrying element has at least two second retaining devices. In one embodiment, the carrying element has a second retaining device on each of the two ends thereof, wherein each is designed to be detachably connectable to a first retaining device on the back element and/or on the shoulder element. Thus, in one embodiment the first retaining devices are detachably connectable to second retaining devices, so that carrying elements may be attached to the stabilising device. In one embodiment, the second retaining devices are configured as hooks, spring clips for example, or as snap fasteners. The detachable connection is made for example by hooking the second retaining device into the first retaining device.

In one embodiment, the carrying element is made from a flexible, stretchable material. The animal owner is thus advantageously able to remain upright when lifting or walking the dog, thereby reducing strain on the users back.

In a preferred embodiment, the stabilising device comprises a front part. In a preferred embodiment, the front part includes a chest element. The chest element is preferably designed such that it may be arranged in the region of the animal's chest.

The term chest element is also referred to as a chest part, for example.

In a preferred embodiment, the chest element is flat. For the purposes of the invention, a flat chest element design is understood to mean that the chest element covers a large expanse of the animal's chest region.

In one embodiment, the chest element includes a flexible, anatomically adaptable supporting area. For the purposes of the invention, the phrase anatomically adaptable means that the chest element is flexible enough to fit snugly against the animal's chest region. This increases carrying comfort. Also advantageously, such a design of the chest element serves to ensure that the animal's musculature, joints and vascular system for example are not subjected to undue strain. In this way, the animal's musculoskeletal system is treated gently.

In one embodiment, the supporting area of the chest element is adapted individually to the animal's chest region. In one embodiment, the supporting area of the chest element is shaped like a tuning fork seen from above, wherein the two tines of the tuning fork pass around the animal's neck.

In one embodiment, at least two connector elements are arranged on the chest element. In a further embodiment, two connector elements are arranged on the chest element. In one embodiment, the connector elements are arranged punctually, i.e. at different positions on the chest element. In one embodiment, the connector elements are arranged at a distance from each other on the chest element. In one embodiment, the connector elements on the chest element do not touch each other. This advantageously ensures a high degree of stability for the stabilising device.

In one embodiment the connector elements are arranged on the chest element so as to be attached non-detachably, by sewing, for example.

In one embodiment, the connector elements are arranged on the chest element mirror symmetrically to the longitudinal axis of the chest element. In this context, the longitudinal axis of the chest element on the chest element represents a notional centrally arranged line in the running direction of the animal. In a preferred embodiment, one connector element is arranged on the left side of the chest element and one connector element is arranged on the right side of the chest element relative to the longitudinal axis. The symmetrical arrangement of the connector elements when the chest and back shoulder elements are connected advantageously ensures a stable, firm and slip-proof grip on the stabilising device.

In one embodiment, first closure means are arranged on the at least two connector elements of the chest element. In one embodiment, at least two first closure means are arranged on the at least two connector elements of the chest element.

In one embodiment, the number of connector elements on the chest element is equal to the number of the first closure means of the chest element. Thus each connector element of the chest element advantageously has a first closure means. In one embodiment, two first closure means are arranged on the two connector elements of the chest element. In one embodiment, two first closure means are arranged on the two connector elements of the chest element. In one embodiment. a first closure means is arranged on each connector element of the chest element.

In one embodiment, the first closure means of the chest element comprise mechanical closure means such as buckles, particularly roller buckles, double pin-buckles, triple pin-buckles, ladder-lock buckles, sliding buckles, clamping buckles, click buckles (quick or click closures, also called click clasps), plug-in buckles or interlocking buckles. In a further embodiment, the first closure means of the chest element comprise mechanical closure means such as toggle closures, hook and loop closures, press studs or zip fasteners. In a further embodiment, the first closure means of the chest element comprise mechanical closure means such as plug locks. In a further embodiment, the first closure means of the chest element include sliding bars, which advantageously enable the length of the connector elements to the shoulder element to be adjusted.

In one embodiment, the connector elements are at least partly arranged on the edges of the chest element. In a further embodiment, the connector elements are arranged on the edges of the chest element, mirror symmetrically to the longitudinal axis of the chest element. In a further embodiment, all connector elements are arranged on the edges of the chest element.

In a preferred embodiment, the stabilising device comprises a front part. In a preferred embodiment, the front part includes a shoulder element. The shoulder element is preferably designed so that it can be fitted on the shoulder region of the animal.

The term shoulder element is also referred to as a shoulder part, for example.

In a preferred embodiment, the shoulder element is of substantially flat design. For the purposes of the invention, the phrase substantially flat design of the shoulder element is understood to describe a shoulder element that covers extensive regions of the shoulder.

In one embodiment, the shoulder element is designed to have a stabilising effect. In one embodiment, the shoulder element has a stabilising effect and is rigid in the region of the spine. In this way, the spine is advantageously supported and prevented from sagging.

In one embodiment, the supporting area of the shoulder element is adapted individually to the animal's shoulder region. In one embodiment, the supporting area of the shoulder element is shaped like a tuning fork seen from above, wherein the two ends of the tuning fork tines pass around the animal's neck.

In one embodiment, the shoulder element has corresponding second closure means for the first closure means of the chest element. In one embodiment, the number of corresponding second closure means of the shoulder element is equal to the number der first closure means of the chest element. In one embodiment, at least two second corresponding closure means are arranged on the shoulder element for the first closure means of the chest element. In a further embodiment, two second corresponding closure means are arranged on the shoulder element for the first closure means of the chest element.

In this context, the corresponding second closure means are arranged on the shoulder element in such manner that the user is easily able to connect the first closure means of the chest element.

Both the first closure means and the corresponding second closure means are arranged on the chest and shoulder elements in such manner that they can easily be accessed by the user.

In one embodiment, at least a portion of the shoulder element to the longitudinal axis includes a stabilising means. In an alternative embodiment, at least a portion of the shoulder element to the longitudinal axis has a plurality of stabilising means. The stabilising means are then advantageously arranged parallel to and at a distance from each other to assure good stability of the shoulder element.

In a further embodiment, the stabilising means extends continuously to the longitudinal axis of the shoulder element. In an alternative embodiment, multiple stabilising means extend continuously to the longitudinal axis of the shoulder element. The stabilising means are then advantageously arranged parallel to the longitudinal axis and at a distance from each other to assure good stability of the shoulder element.

In this context, the longitudinal axis of the shoulder element represents the notional centrally arranged line in the running direction of the animal. The longitudinal axis of the shoulder element extends along the animal's spine.

In one embodiment, the stabilising means in the shoulder element is a panel or brace. In one embodiment, the stabilising means of the shoulder element is introduced in a cutout provided for this purpose on the upper surface of the shoulder element. In a further embodiment, the stabilising means of the shoulder element is fastened inside the shoulder element, by sewing for example. In a further embodiment, the stabilising means of the shoulder element is sewn underneath a connector element of the shoulder element.

In one embodiment, the stabilising means in the shoulder element is a metal brace. In a further embodiment the stabilising means in the shoulder element is a plastic brace, the advantage of which is that the weight of the shoulder element is reduced thereby.

Inserts and seams serve to stabilise the shoulder element further.

In one embodiment, the chest element is connected to the shoulder element via at least one detachable connector.

A detachable connector between the chest element and the shoulder element may also be understood to refer to a seam in the connection between the shoulder element and the chest element for example.

In one embodiment, the chest element is designed to be detachably connectable to the shoulder element via the connector elements located on the chest element, with the first closure means arranged thereon and the corresponding second closure means arranged on the shoulder element. In a preferred embodiment, the chest element is connected to the shoulder element by at least two connector elements. In this context, the first closure means of the chest element is designed to be connectable with the second closure means of the shoulder element in such manner that it can be detached mechanically.

In a further embodiment, the connector elements of the chest element and the second closure means of the shoulder element corresponding to the first closure means are in the form of hook and loop closures which are detachably connectable to each other.

When the chest element and the shoulder element are connected to each other, in one embodiment the connector elements extend along anatomically suitable positions of the animal's rump. Anatomically suitable positions according to the invention are understood to be those positions that do not hinder or hurt the animal when it moves. These include for example the course of the connector elements in the region of suitable ribs, particularly in the region of the third, fourth or fifth rib. When connected, the connector elements also pass between the chest element and the shoulder element without straining the muscles or constricting any blood vessels.

In a preferred embodiment, the stabilising device comprises a device for acquiring vital functions of animals and/or for locating and/or monitoring and/or transmitting geodata of animals.

In one embodiment, at least some of the components and/or some of the connector elements of the stabilising device include textile materials.

In one embodiment, the components of the stabilising device have an outer layer. In a further embodiment, the components of the stabilising device have an inner layer. The outer layer corresponds to the layer which is in contact with the environment surrounding the animal. The inner layer corresponds to the layer which is in contact with the animal's skin or coat. In a further embodiment, the components of the stabilising device include an intermediate layer. The intermediate layer corresponds to a filling which is arranged between the outer layer and the inner layer.

In one embodiment, all parts of the stabilising device that include textile materials comprise the same textile material. Advantageously, the textile material is robust. In an alternative embodiment, all parts of the stabilising device that include textile materials comprise different textile materials.

In one embodiment, the robust textile material is a fabric or cloth. In one embodiment, the robust textile material is a fabric consisting of synthetic and/or natural fibres. In one embodiment, the robust textile material is a fabric made from polyamide fibres. In one embodiment, the robust textile material is a fabric made from polyamide fibres and various other synthetic or natural fibres. In one embodiment the robust textile material is a textile material which is also used as the outer fabric of suitcases and will be referred to as suitcase fabric in the following text. The robust textile material of the stabilising device is characterised by advantageous properties: for example, the robust textile material is dirt-repellent, washable and breathable. The robust textile material advantageously dries quickly, is durable and easy to clean.

In one embodiment, the robust textile material has a water column of 3000 to 4000 mmH₂O, preferably 3500 mmH₂O. In a further embodiment, the robust textile material of the stabilising device exhibits tensile strengths in the range from 1 to 5 kg/cm. The tensile strength of the robust textile material is advantageously selected on the basis of the size of the dog and thus also on the basis of the size of the respective belly, chest, shoulder and back elements.

In one embodiment, at least some of the components of the stabilising device include an intermediate layer. In one embodiment, the belly element and/or the chest element and/or the shoulder element and/or the back element has/have an intermediate layer. In a further embodiment, the supporting area of the belly element and/or chest element includes an intermediate layer.

In one embodiment, the intermediate layer is a textile material. In one embodiment, the intermediate layer has the form of a textile fabric and is interposed, for example woven in between the textile material of the outer and/or inner layer. In one embodiment, the intermediate has the form of a spacer fabric. The spacer fabric is non-elastic.

In a preferred embodiment, at least some of the components of the stabilising device include a non-elastic spacer fabric. In one embodiment, the non-elastic spacer fabric at least partly consists of cotton. In a further preferred embodiment, the spacer fabric is non-elastic and has low stretchability. In a preferred embodiment, the stretchability of the non-elastic spacer fabric is between 0% and 15%, preferably between 0% and 10%, particularly preferably between 0% and 5%, most particularly preferably around 0%. The spacer fabric advantageously assures the stability of the belly element, the back element, the shoulder element and/or the chest element.

Due to the high degree of stability of the non-elastic spacer fabric, the connector elements extending away from the belly element may advantageously be arranged on a point-by-point basis, not continuously. Due to the high degree of stability of the non-elastic spacer fabric, continuous connector elements that pass through the belly element may advantageously also be omitted. This in turn advantageously increases carrying comfort for the animal, and no body parts, organs, muscles or blood vessels are constricted, compressed or injured in the belly region or in any region where the spacer fabric is inserted.

In one embodiment, the components of the stabilising device are at least partly padded. In one embodiment, at least parts of the belly element and/or the back element and/or the chest element and/or the shoulder element are padded. In one embodiment, the chest element includes a sternum padding.

In one embodiment, the components of the stabilising device are padded. In one embodiment, the belly element and/or the back element and/or the chest element and/or the shoulder element are padded. In one embodiment, the chest element includes a sternum padding.

In a further embodiment, the connector elements are padded. In a further embodiment, the components and connector elements of the stabilising device are padded. In a further embodiment, at least some of the components and connector element of the stabilising device are padded.

In one embodiment, the connector elements of the components and/or the connectors of the detachable connecting means are made from a mixture of polypropylene and polyester, preferably from a mixture of 83% polypropylene and 17% polyester. These have a maximum tensile strength of at least 4500 N (approx. 450 kg) and have a width of about. 25 mm, a thickness of about 1.19 mm, and a weight of about 1.31 kg/100 m.

In an alternative embodiment, the connector elements of the components or the connectors of the detachable connecting means are made of polyester, preferably 100% polyester. They have a maximum tensile strength of at least 7500 N (approx. 750 kg) and have a width of about 40 mm, a thickness of about 1.80 mm and a weight of about 4.62 kg/100 m.

In one embodiment, the first and second closure means of the components of the stabilising device or the first and second closure means of the detachable connecting means of the stabilising device are made of metal or plastic.

In one embodiment, the plug locks are made of polyamide, preferably polyamide 6.6, have a strength of 160 kg, 280 kg or 600 kg and a mesh width of 40 mm auf. Some plug locks also have an adjustment capability as well a strength of 105 kg and mesh width of 40 mm.

In one embodiment, the spring clips are made of metal or plastic. The spring clips are preferably made of acetal, and have a strength of 35 kg and a mesh width of 25 mm.

In one embodiment, the sliding bars are made of metal or plastic. In a further embodiment, the sliding bars are made of nylon and have a mesh width of 40 mm. The sliding bars are not directly exposed to tensile load, because the load is distributed through the connector elements of the components and through the connectors of the detachable connecting means.

In a preferred embodiment, a temperature control device is installed in at least one component of the stabilising device.

In one embodiment, the temperature control device is designed so that it may be heated and/or cooled.

In a preferred embodiment, a temperature control device is at least partly installed in at least one component of the stabilising device. In one embodiment, a temperature control device is at least partly installed in the belly element and/or the back element and/or the chest element and/or the shoulder element.

In one embodiment, the temperature control device is installed in the intermediate layer of the components of the stabilising device. In one embodiment, the temperature control device comprises a Peltier element, which enables the stabilising device to be heated or cooled. In a further embodiment, the temperature control device comprises heating wires and/or heater pads and/or cooling storage cells and/or cooling gels.

In one embodiment, the animal may advantageously be warmed and/or protected from hypothermia with a temperature control device that can be heated and/or cooled. The heatable temperature control device enables the animal to be kept advantageously in a transport means even at lower temperatures.

In one embodiment, the animal may advantageously be cooled and/or protected from overheating with a coolable temperature control device. The coolable temperature control device enables the animal to be kept advantageously in a transport means even at higher temperatures.

In one embodiment, the stabilising device is heated or cooled by electrical and/or chemical exothermic processes.

In one embodiment, the energy for the temperature control device is supplied by an energy storage device fitted in the stabilising device.

In one embodiment, the belly element with the connector elements and the first closure means arranged thereon as well as the back element with the second closure means corresponding to the first closure means of the belly element form the rear part. In a preferred embodiment, the stabilising device according to the invention comprises a front part. In one embodiment, the chest element and the shoulder element form the front part.

In a preferred embodiment, the front part includes flat chest element, which is designed to be fitted in the chest region, and a flat shoulder element, which is designed to be fitted in the shoulder region.

In one embodiment, the rear part and the front part are detachably connected to each other.

In one embodiment, the rear part and the front part are connectable with each other via at least one detachable connecting means.

The detachable connecting means either connects the belly element to the chest element and/or the back element to the shoulder element (a detachable connecting means between the belly element and the shoulder element or between the back element and the chest element is not possible). In a preferred embodiment, the belly element includes a detachable connecting means with the chest element. In a further embodiment, the back element includes a detachable connecting means with the shoulder element.

In a further embodiment, the rear part and front part are connected to each other via two detachable connecting means. The belly element has a detachable connecting means with the chest element and the back element has a detachable connecting means with the shoulder element, wherein the detachable connecting means are detachably connected to the chest element and the shoulder element respectively.

In one embodiment, the detachable connecting means includes at least one first and at least one second closure means and a connector.

In one embodiment, the first closure means of the detachable connecting means between the rear part and the front part comprise mechanical closure means such as buckles, particularly roller buckles, double pin buckles, triple pin buckles, ladder-lock buckles, sliding buckles, clamping buckles, click buckles (quick release or click closure, also called click clasps), plug-in buckles, or interlocking buckles. In a further embodiment, the first closure means of the detachable connecting means between the rear part and the front part comprise mechanical closure means such as toggle closures, hook and loop closures, press studs or zip fasteners. In a further embodiment the first closure means of the detachable connecting means comprise mechanical closure means such as plug locks. In a further embodiment, the first closure means of the detachable connecting means is a spring clips with a corresponding counterpiece as the second closure means of the detachable connecting means. In a further embodiment, the first closure means of the detachable connecting means include sliding bars, which are advantageous in that they enable the length of the connector between the front part and the rear part to be adjusted.

In one embodiment, a corresponding second closure means is arranged on the front part to accommodate the first closure means of the detachable connecting means of the rear part.

In one embodiment, the second closure means of the detachable connecting means function as the corresponding counterpieces for the first closure means of the detachable connecting means, so that the first and second closure means are advantageously designed to be detachably connectable with each other in positive locking manner.

In one embodiment, the chest element or the belly element includes a corresponding second closure means for accommodating the first closure means of the detachable connecting means of the belly element or the chest element. In a further embodiment, the back element or the shoulder element has a corresponding second closure means for accommodating the first closure means of the detachable connecting means from the shoulder element or the back element.

The second closure means of the detachable connecting means are advantageously arranged on the chest element or belly element in such manner that the user is easily able to reach them and connect them to the first closure means of the detachable connecting means of the belly element or the chest element.

In one embodiment, the first or second closure means of the detachable connecting means are arranged punctually on the belly element and/or the back element. In a further embodiment, the first and the second closure means of the detachable connecting means are arranged punctually to the longitudinal axis of the belly element and/or of the back element. In a further embodiment, the first or second closure means of the detachable connecting means are arranged punctually on one end of the longitudinal axis of the belly element or of the back element.

In one embodiment, the connector of the detachable connecting means on the rear part is arranged so as to be connected non-detachably, by sewing for example. In a further embodiment, the connector of the detachable connecting means on the belly element and/or on the back element is/are arranged so as to be connected non-detachably, by sewing for example.

In one embodiment, the connector of the detachable connecting means on the front part is arranged so as to be connected non-detachably, by sewing for example. In a further embodiment, the connector of the detachable connecting means on the chest element and/or on the shoulder element is/are arranged so as to be connected non-detachably, by sewing for example.

In one embodiment, the connector of the detachable connecting means is realised as a belt or strap. In a preferred embodiment, the connector of the detachable connecting means is designed as a belt strap. In a further embodiment, the connector of the detachable connecting means has adjustable length and width.

In one embodiment, the stabilising device further comprises a bag.

In one embodiment, the bag holds the stabilising device for transporting it.

In one embodiment, the bag is configured as a rucksack and/or a pouch.

In one embodiment, the bag has an opening means, such as a zip fastener for example. In order to open the bag, the opening means is opened and the stabilising device can be placed inside the bag. The stabilising device can advantageously be transported in this way.

In a preferred embodiment, the bag includes an integral carrying element. In a further preferred embodiment, the carrying element is designed to be detachable from the bag and may be fastened thereto as needed at suitable first retaining devices on the stabilising device. For this purpose, the carrying element for the bag has a second retaining device. The carrying element and the first retaining device of the bag are designed similarly to the carrying element and the first retaining device of the stabilising device. This enables the bag to be transported conveniently by the user. The carrying element for the bag may also be fastened detachably to the stabilising device.

In a preferred embodiment, the bag is designed to be usable as a blanket. For this purpose, the bag is opened, the stabilising device is taken out and the bag is spread flat for use as a blanket. The area of the spread out bag when open is advantageously at least equal to the area of the supporting area for the animal, which is then placed on the blanket created in this way.

In one embodiment, the bag includes connector elements which are connected non-detachably inside it. In a preferred embodiment, detachable first closure means are arranged on the connector elements which are connected non-detachably to the bag. In a further preferred embodiment, the bag includes second closure means corresponding to the first closure means and fastened to the bag.

In a preferred embodiment, the bag is designed to be usable as a weather covering, such as a rain cape for example. For this, the bag is opened, the stabilising device is removed and the opened bag is laid flat on the animal's back. The connector elements connected non-detachably in the bag are wrapped around the animal's rump and fastened with the detachable first closure means. The area of the unfolded bag is advantageously at least equal to that of the supporting area on the animal's back, on which the weather covering created in this way is laid. The detachable first closure means of the bag and the corresponding second closure means are configured similarly to the detachable first closure means of the stabilising device and the corresponding second closure means. The animal can then advantageously carry the stabilising device according to the invention itself when the opened bag is placed over it in the manner of a rain cape.

In one embodiment, at least a part of the bag is made from a textile material. In one embodiment, the bag is made entirely from a textile material. The textile material is advantageously designed to be robust. In one embodiment, the robust material of the bag is the same robust textile material is used for the stabilising device.

In one embodiment, at least a part of the bag is padded. In one embodiment, the bag is completely padded. In a preferred embodiment, the bag is adapted to the size of the components of the stabilising device and thus also to the size of the animal. In one embodiment, the bag is adapted to the size of the components and the connector elements of the stabilising device ad thus also to the size of the animal.

In a preferred embodiment, at least part of the bag included a fitted temperature control device.

In a preferred embodiment, the stabilising device according to the invention is used for medical treatment, relieving strain, stabilising and securing the animal as well as lifting it horizontally and carrying it.

The stabilising device according to the invention fits snugly against anatomically appropriate and suitable body parts of the animal. For the purposes of the invention, anatomically appropriate and suitable positions are understood to be those positions which do not hinder or hurt the animal when it moves.

In one embodiment, the components of the stabilising device according to the invention are configurable individually in variable and various dimensions. In this way, the size of the components may also be selected to match the size of the corresponding body parts.

The objective is to relieve the animal's musculoskeletal system of stress. This system particularly includes its limbs, joints, tendons, muscles and the spine. The spine is not twisted when the animal is lifted up, instead it is held in place laterally and stabilised, so that undesirable strain and twisting of the animal's rump is prevented.

The stabilising device according to the invention advantageously does not pinch any muscles or blood vessels. This prevents any pressure from being applied to regions or punctual sites on the animal's body, which increases the feeling of comfort when the stabilising device is carried.

Carrying comfort is increased for the animal itself. Also advantageously, the shape of the supporting area of the belly element further ensures handling of the animal that is gentler on the muscles, joints and vascular system. In this way, the animal's musculoskeletal system is treated gently.

The stabilising device according to the invention further enables the user or the person holding the animal to adopt a proper posture. The configuration of the stabilising device is such that it allows the holder to adopt a correct posture and walk upright when lifting the animal or walking with it, which reduces strain on the owner's own back.

The stabilising device according to the invention also provides overall support for older animals. This applies particularly to animals which are no longer able to walk for long distances due to their age.

The stabilising device according to the invention is also designed to be usable after injuries or diseases of the muscles, limbs and joints. The animal can be held in place firmly and stably with the stabilising device according to the invention. This is also possible for a resting position. Because it can be disassembled into its individual parts, the stabilising device according to the invention may also be placed over an animal which is lying down easily and without difficulty, without the animal having to move or step through an opening. Such a condition may be due to injury, or also if the animal has been anaesthetised but must still be moved or transported. The stabilising device according to the invention is thus easily operable by a user without disturbing the animal unnecessarily.

A further beneficial reason for the use the stabilising device according to the invention in medical situations may be seen after operations from which the animals must recover. After an operation, animals are often weakened and their anatomy is changed, for example due to loss of muscle mass from weight loss following an operation. In this situation, it is beneficial to have recourse the precisely fitting, anatomically gentle stabilising device which can be adapted individually to the existing anatomical proportions and can be used as a working aid without straining the muscles unduly.

With the stabilising device according to the invention the animal can be lifted up and carried practically horizontally. In this way, assistance can be provided in the short or longer term for climbing stairs or negotiating obstacles such as door thresholds for example. For the purposes of the invention, a horizontal position of the animal refers to the alignment of the rump perpendicularly to the force of gravity. Thus, the animal's rump is parallel to the ground, which reflects the natural attitude of the animal when standing. Thus, the animal's natural instinct always to stand with all four legs on the ground is satisfied.

Users of the stabilising device according to the invention are for example dog owners who wish to use the stabilising device when walking their pets or who must pick their dog up and carry it after an operation or due to advancing age.

The stabilising device according to the invention may also be used outdoors. Furthermore, the stabilising device according to the invention may also be used by rescue organisations, including mountain rescue, the police, the fire brigade, emergency services or other recovery organisations for example in impassable terrain, e.g., in high mountains as a guidance or search and rescue harness for rescue dogs. The stabilising device according to the invention may also be used with animals used by the military or hunting animals, such as hunting dogs. The stabilising device according to the invention may also be used by extreme athletes such as climbers in impassable terrain, in high mountains for example. In this situation, the animal, a dog for example, may be lowered by rope without undue physical stress because it remains practically horizontal. The animal also remains calm, because this practically horizontal position also corresponds to its natural standing position (all four legs pointing towards the ground). The animal is also stabilised by the stabilising device. Furthermore, the stabilising device according to the invention can be fitted and removed easily and quickly.

All other animal owners are also considered to be potential users of the stabilising device according to the invention.

In addition, veterinarians, animal rehabilitation clinics or animal therapy schools where animals which have recently undergone surgery are monitored and are retrained in water baths for example are also considered to be potential users of the stabilising device according to the invention. At the same time, the stabilising device according to the invention may also be worn even in water to relieve the burden on a part of the muscular system.

For the purpose of realising the invention, it is also beneficial to combine the variants and embodiments according the invention described previously and the features of the claims in any order.

### EXEMPLARY EMBODIMENTS

In the following text, the invention will be explained in greater detail on the basis of an exemplary embodiment. The embodiment relates to a stabilising device for dogs and is intended to describe the invention without any limitation thereof.

The stabilising device for dogs comprises a belly element, a back element, a chest element and a shoulder element. The individual elements are connected to each other with straps.

The invention will be explained in greater detail with reference to drawings. The drawing shows:

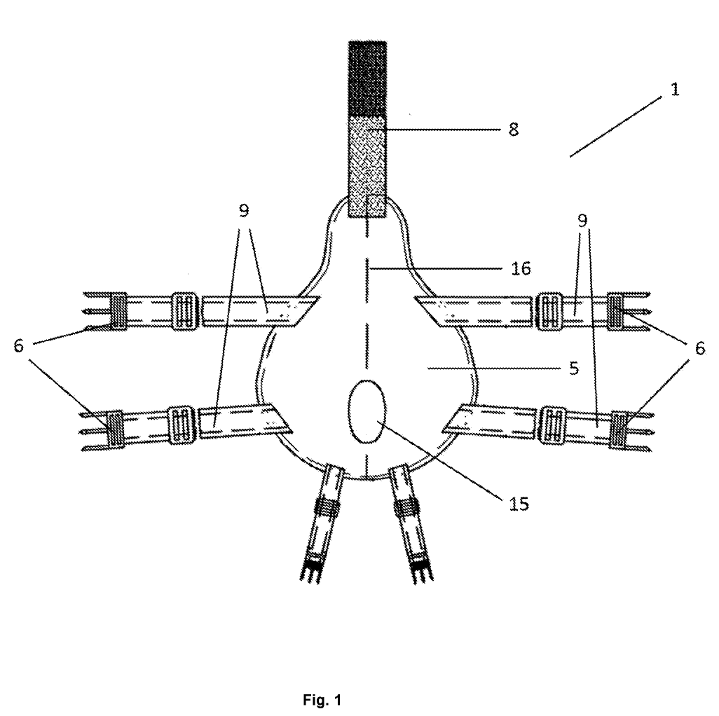

FIG. 1 Belly element of the stabilising device for dogs

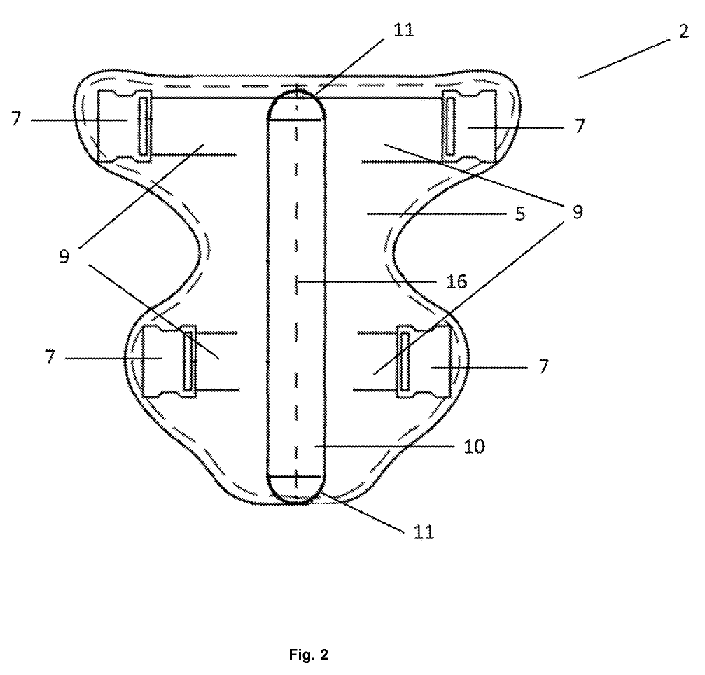

FIG. 2 Back element of the stabilising device for dogs

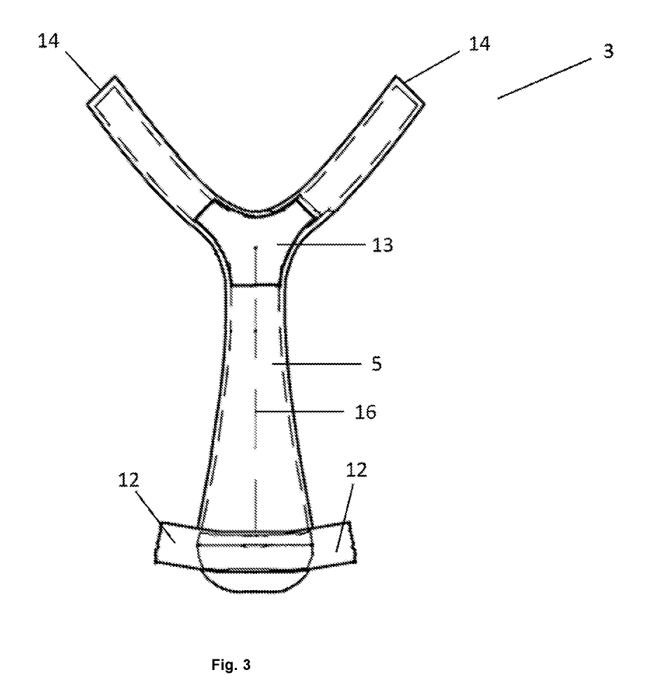

FIG. 3 Chest element of the stabilising device for dogs

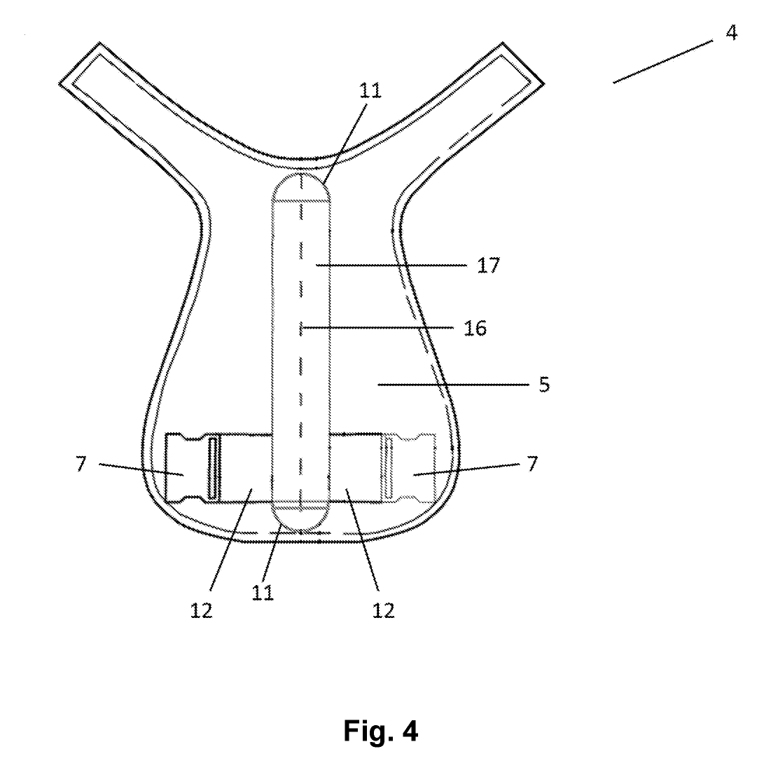

FIG. 4 Shoulder element of the stabilising device for dogs

The upper parts of the components depicted on the figures each point toward the head of the dog, while the lower parts of the components point toward the tail of the dog. The respectively longitudinal axes of the components recorded from the top down run along the spine of the dog, and relate to its running direction.

FIG. 1 shows a belly element 1 with a pear-shaped supporting area 5.

The belly element 1 has four belt straps 9 arranged punctually thereon, which are arranged mirror symmetrically to the longitudinal axis 16 of belly element 1 and at a distance from each other. Two belt straps 9 are arranged on the left side and two belt straps 9 are arranged on the right side of supporting area 5 of the belly element 1 relative to the longitudinal axis 16. In addition, the four punctually arranged belt straps 9, two narrower belt straps 9 are shown on the lower side of the belly element 1 in the figure, through which the dog's tail is guided. The length of all belt straps 9 can be adjusted with sliding bars.

The cutout 15 to allow the sex organ of a male dog to guide through is provided centrally and symmetrically to the longitudinal axis 16 in the belly element 1.

The belt straps 9 are sewn to the belly element. A click buckles 6 is provided on each belt strap 9 through which the belly element 1 is connected to the back element 2 (connector and click buckles not shown in the figure).

The top of the belly element 1 is equipped with a connecting strap 8 as a detachable connecting means which extends parallel to the longitudinal axis 16 and by which the belly element 1 is connected to a chest element 3 (chest element and connector not shown in the figure). The connecting strap 8 is sewn to the belly element 1 in the same way as the belt straps 9.

The belly element 1 is padded and includes a spacer fabric (not shown in the figure).

FIG. 2 shows a back element 2 with a flat supporting area 5. The back element 2 has four belt straps 9, which are sewn onto the back element 2 parallel, perpendicular and at a distance from each other relative to the longitudinal axis 16 and arranged mirror symmetrically to the longitudinal axis 16. A counterpiece 7 for each click buckle 6 attached to the belly element 1 is arranged on each belt strap 9, and by which the belly element 1 is detachably connected to the back element 2 (connector not shown in the figure).

A back brace 10 made of plastic is fastened to the back element 2, and is sewn underneath the belt straps 9 which extend over the back brace 10 of the back element 2. The back brace 10 extends continuously along the longitudinal axis 16 of the back element 2, and thus also along the dog's spine as far as to the head of its tail.

The back element 2 further has a D-ring 11 at both its upper and lower ends along the longitudinal axis 16. The D-rings 11 are each sewn to a belt strap 9 at the two most distant points of the back element 2 along the longitudinal axis 16. The end of a carrying handle may be clipped into each D-ring 11 (not shown in the figure).

The back element 2 is padded (not shown in the figure).

FIG. 3 shows a chest element 3 which has a supporting area 5 in the shape of a tuning fork.

The bottom of the chest element 3 has two belt straps 12 arranged punctually on the chest element 3, each of which is arranged mirror symmetrically to the longitudinal axis 16 of the chest element 3. The belt straps 12 extend continuously over the chest element 3. One click buckle (not shown in the figure) arranged thereon is attached to each belt strap 12.

The chest element 3 has a detachable connector 14 at the ends of each of the two tines of the tuning fork-shaped supporting area 5, by which connectors the chest element 3 is detachably connected with the shoulder element 4 (shoulder element not shown in the figure).

The bottom of the chest element 3 has a hook and loop closure for the connecting strap 8 coming from the belly element 1, by which the chest element 3 is connected to a belly element 1 (connecting strap and hook and loop closure not shown in the figure). The connecting strap 8 is also sewn to the belly element 1 in the same way as the belt straps (9 and 12).

The chest element 3 has a sternum padding 13 and a spacer fabric (spacer fabric not shown in the figure).

FIG. 4 shows a shoulder element 4, which has a tuning fork-shaped supporting area 5 which is wider in design than the surface of the chest element 3.

The bottom of the shoulder element 4 has two belt straps 12 arranged punctually on the shoulder element 4, each of which is arranged mirror symmetrically to the longitudinal axis 16 of the shoulder element 4. The belt straps 12 extend continuously over the shoulder element 4. A counterpiece 7 for each click buckle 6 attached to the chest element 3 is arranged on each belt strap 12, and by which the chest element 3 is detachably connected to the shoulder element 4 (chest element and connector not shown in the figure).

The shoulder element further has a click closure at the ends of its two tines, by which the shoulder element is connected detachably to the chest element (not shown in the figure).

A shoulder brace 17 made of plastic is fastened to the shoulder element 4, and is sewn underneath the belt straps 12 which extend over the shoulder brace 17 of the shoulder element 4. The shoulder brace 17 extends continuously along the longitudinal axis 16 of the shoulder element 4 and thus also along the dog's spine.

The shoulder element 4 further has a D-ring 11 at both its upper and lower ends along the longitudinal axis 16. The D-rings 11 are each sewn to a belt strap 12 at the two most distant points of the shoulder element 4 along the longitudinal axis 16. The end of a carrying handle may be clipped into each D-ring 11 (not shown in the figure). The two ends of the carrying handle may also be clipped into a D-ring on the shoulder element 4 and a D-ring 11 on the back element 2, so that the dog may be carried both by the shoulder element 4 and by the back element 2.

The shoulder element 4 is padded (not shown in the figure).

### REFERENCE SIGNS

1 Belly element
2 Back element
3 Chest element
4 Shoulder element
5 Supporting area
6 Click buckle
7 Click buckle counterpiece
8 Detachable connecting strap (on the belly element) with the chest element
9 Detachable belt strap (on the belly element) with the back element
10 Back brace
11 D-rings for attaching carrying handles
12 Detachable belt strap (on the chest element) with the shoulder element
13 Sternum padding
14 Detachable connector (on the chest element) with the shoulder element
15 Cutout for a male dog's sex organs
16 Longitudinal axis
17 Shoulder brace

1. Stabilising device for animals for medical treatment, relieving strain, stabilising, securing, and for horizontal lifting and carrying, comprising at least the following components:
a substantially flat belly element, which
is designed to be attached in the belly region, having
a) a flexible, anatomically adaptable supporting area and
b) at least four connector elements with first closure means arranged thereon,
and a flat back element which
is designed to be attached in the back region, and has
c) at least one stabilising means aligned at least in sections to the longitudinal axis, and
d) at least four second closure means,
wherein
the at least four connector elements are arranged on the belly element at a distance from each other mirror symmetrically to the longitudinal axis of the belly element,
the belly element is designed to be detachably connectable to the back element via the connector elements with the first closure means arranged thereon and the corresponding second closure means, wherein the number of first closure means is equal to the number of corresponding second closure means.

2. Stabilising device for animals according to claim 1, characterised in that at least a part of the components of the stabilising device has a non-elastic spacer fabric with an elasticity between 0% and 15%.

3. Stabilising device for animals according to claim 1, further comprising a front part, which includes a flat chest element, which is designed to be attached in the chest region, and a flat shoulder element, which is designed to be attached in the shoulder region.

4. Stabilising device for animals according to claim 1, characterised in that the chest element is connected to the shoulder element via at least two connector elements.

5. Stabilising device for animals according to claim 1, characterised in that the belly element has a detachable connecting means to the chest element.

6. Stabilising device for animals according to claim 1, characterised in that it has at least two first retaining devices, wherein the first retaining devices are arranged at a distance from each other on the back element and/or on the shoulder element.

7. Stabilising device for animals according to claim 1, characterised in that at least one detachable carrying element, which has at least two second retaining devices, can be inserted in the first retaining devices.

8. Stabilising device for animals according to claim 1, characterised in that a temperature control device is fitted in at least a part of at least one component.

9. Stabilising device for animals according to claim 1, characterised in that the belly element has a cutout for the sex organ of the male animal.

10. Stabilising device for animals according to claim 1, further comprising a device for detecting vital functions of animals and/or for locating and/or monitoring and/or transmitting geodata of animals.

11. Bag for holding a stabilising device for animals according to claim 1, characterised in that the bag has an integrated, detachable carrying element and connector elements first closure means arranged thereon, and corresponding second closure means, and is designed to be usable as a blanket or a cape with a temperature control device fitted in at least a part thereof.

12. Use of a stabilising device for animals according to claim 1 for medical treatment, relieving strain, stabilising, securing and for horizontal lifting and carrying animals.

13. Use of a bag according to claim 11 for holding a stabilising device, wherein said stabilising device is for animals for medical treatment, relieving strain, stabilising, securing, and for horizontal lifting and carrying, comprising at least the following components:
a substantially flat belly element, which
is designed to be attached in the belly region, having
a) a flexible, anatomically adaptable supporting area and
b) at least four connector elements with first closure means arranged thereon,
and a flat back element which
is designed to be attached in the back region, and has
c) at least one stabilising means aligned at least in sections to the longitudinal axis, and
d) at least four second closure means,
wherein
the at least four connector elements are arranged on the belly element at a distance from each other mirror symmetrically to the longitudinal axis of the belly element,
the belly element is designed to be detachably connectable to the back element via the connector elements with the first closure means arranged thereon and the corresponding second closure means, wherein the number of first closure means is equal to the number of corresponding second closure means.
